(19) United States
(12) Patent Application Publication
(10) Pub. No.: US 2025/0384997 A1
(43) Pub. Date: Dec. 18, 2025

(54) AUTOMATED ASSET IDENTIFICATION SYSTEM

(71) Applicant: CareFusion 303, Inc., San Diego, CA (US)

(72) Inventors: Michael K. WORKMAN, Calrsbad, CA (US); Joshua Jay HARMAN, Seguin, TX (US)

(21) Appl. No.: 19/130,329

(22) PCT Filed: Nov. 15, 2022
(86) PCT No.: PCT/US2022/049995

§ 371 Date: May 15, 2025

(51) Int. Cl. G16H 40/40

(52) U.S. Cl. CPC G16H 40/40

(57) ABSTRACT

An automated asset identification system and method is disclosed. A request to create an identification template is received for a medical device based on a first identifier currently assigned to the medical device and provisioning information obtained from an identification server, and the provisioning information is received based on the request. The identification template is generated based on the first identifier and the provisioning information and, responsive to generating the identification template, a visualization of the identification template is provided to the medical device for display on a display screen of the medical device.

### BACKGROUND

This application relates generally to ensuring that an infusion device is properly identified.

Modern infusion devices are mobile and able to administer medications to a patient as the patient moves between care areas throughout a medical environment such a hospital organization. Hospitals are also coming under increased pressure to contain costs. One effect of this pressure is a need to maintain a current inventory of assets, such as mobile medical devices that are a part of the hospital infrastructure, as well as knowledge of the location of the assets. Hospitals often look to outside vendors to maintain such inventory; however, modern vendors continue to use outdated barcodes for asset identification. These physical barcodes, typically applied directly to the medical devices, degrade or become damaged or destroyed over time, for example due to cleaning or extended use, or merely as a result of age. It becomes difficult to know which devices are assigned to which identifiers stored within a given vendor system.

### SUMMARY

The subject technology provides a system and method for automated asset identification system. Unlike legacy systems and processes, the subject technology allows a third party system to assign its own unique identifiers to assets maintained by a local hospital system. A medical device asset is assigned an identification template which is mapped to the third party identifier and provided to the medical device for display on the display screen. The template itself may be reconfigured to be displayed differently depending on third party requirements.

In this regard, the subject technology includes a medical information system, comprising one or more computing devices configured to: receive a request to create an identification template for a medical device based on a first identifier currently assigned to the medical device and provisioning information obtained from a template server; receive the provisioning information based on the request; generate the identification template based on the first identifier and the provisioning information; and provide to the medical device, responsive to generating the identification template, a visualization of the identification template for display on a display screen of the medical device. Other aspects include corresponding devices, methods, and computer program products for implementation of the corresponding system and its features.

A machine-implemented method comprises receiving a request to create an identification template for a medical device based on a first identifier currently assigned to the medical device and provisioning information obtained from an identification server; receiving the provisioning information based on the request; generating the identification template based on the first identifier and the provisioning information; and providing to the medical device, responsive to generating the identification template, a visualization of the identification template for display on a display screen of the medical device. Other aspects include corresponding devices, systems, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B depicts a closer view of a portion of the example patient care system shown in FIG. 1A, according to various aspects of the subject technology.

FIG. 2 depicts an example identification template for identifying a medical device to multiple different systems together with corresponding example displayable representations, according to aspects of the subject technology.

FIG. 3 depicts an example system for generating and displaying an identification template for a medical device, according to aspects of the subject technology.

FIG. 4 depicts a first example process for an automated asset identification system, according to aspects of the subject technology.

FIG. 5 depicts a second example process for an automated asset identification system, according to aspects of the subject technology.

FIG. 6 is a conceptual diagram illustrating an example electronic system for an automated asset identification system, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Scanning barcodes to identify infusion devices or their connected modules have become problematic, particularly when the barcodes are on the back of the device or have faded over time. The subject technology provides a system for publishing, to specific medical devices, a custom identification template for visually identifying the medical device(s), assigning an identifier to the template, and causing the device to display a virtual representation of the template to be used in place of a physical identifier.

Instead of adding a new physical barcode or other identifier to a medical device for the purpose of scanning or manually entering it later into an external system to identify the device to that system, the medical device can be activated to request, from a server, a supplemental identifier for identifying the medical device to the external system. According to various implementations, the supplemental identifier is particular to the external system for identifying the medical device, for example, across an asset tracking system. The supplemental identifier may be requested by activating a control on the medical device or, in some implementations, using a terminal associated with the external system, separate from the infusion device.

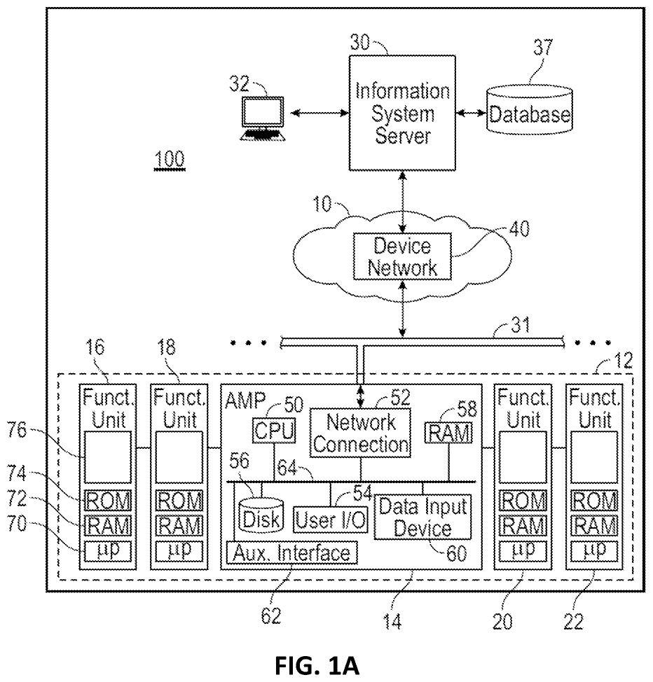

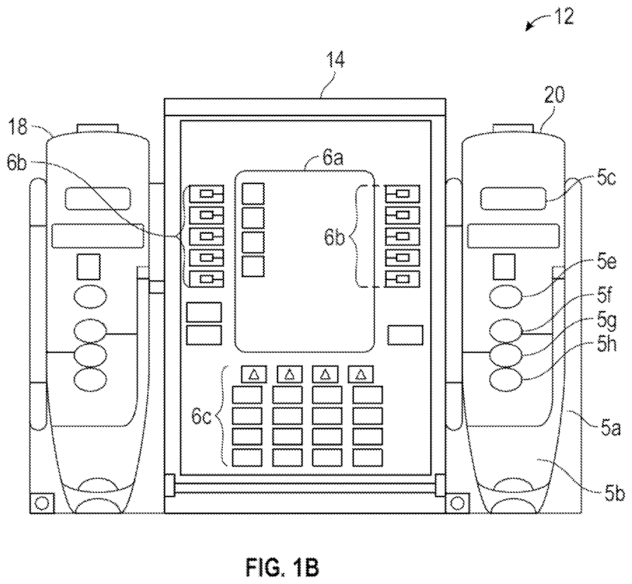

FIG. 1A depicts an example of an institutional patient care system 100 of a healthcare organization, according to aspects of the subject technology. In FIG. 1A, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1A, at least one of functional modules 16, 18, 20, 22 may be an infusion pump module such as an intravenous infusion pump for delivering medication or other fluid to a patient. For the purposes of this discussion, functional module 116 is an infusion pump module. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may be a printer, scanner, bar code reader, near-field communication reader, RFID reader, or any other peripheral input, output or input/output device.

Each functional module 16, 18, 20 and/or 22 communicates directly or indirectly with interface unit 14, with interface unit 14 providing overall monitoring and control of device 12. Functional modules 16, 18, 20 and/or 22 may be connected physically and electronically in serial fashion to one or both ends of interface unit 14 as shown in FIG. 1A, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1A, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 52 (as shown in FIG. 1A), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information system (HIS) server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

According to various implementations, server 30 includes a formulary and/or pharmacy information system. Pharmacy information systems may enable a safer physician medication order process. A pharmacy website (e.g., provided by the server) may provide the physician with a list of available drugs from which the physician may select. The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information system 30. The pharmacy reviews the order, and once the order has been prepared, the order may be transmitted to the nurse station for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The establishment of these parameters, along with parameters for off-formulary orders, via the system 30 is useful for maintaining consistency across the healthcare environment and ensuring an order is intelligible and executed according to expectations by other devices within the system 30 (e.g., an infusion pump).

With further reference to FIG. 1A, patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the interface unit 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display for displaying a user interface, including a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

FIG. 1B is a closer view of a portion of the example patient care system shown in FIG. 1A, according to various aspects of the subject technology. FIG. 1B shows two of the fluid infusion pumps mounted at either side of a programming module, and the displays and control keys of each, with the programming module being capable of programming both infusion pumps. The infusion device includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 20. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 20, such as alert indications (e.g., alarm messages). Control keys 5e-h exist for programming and controlling operations of the infusion pump as desired. In some implementations, the control keys may be presented as interactive elements on the display 5c (e.g., touchscreen display). The infusion device and/or infusion pump may also include audio alert equipment in the form of a speaker (not shown).

The programming module 14 of the infusion device 12 includes a display 6a for visually communicating various information, such as the operating parameters of a connected pump and alert indications and alert messages, and control keys 6b and 6c for selecting and/or setting control parameters and/or options for controlling the infusion device 12 and connected modules. The programming module 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The programming module 14 may include a communications system (not shown) with which the programming module 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). The communication module may be used to transfer access and interaction information for clinicians encountering the programming module or device coupled therewith (e.g., pump 20 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the programming module 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

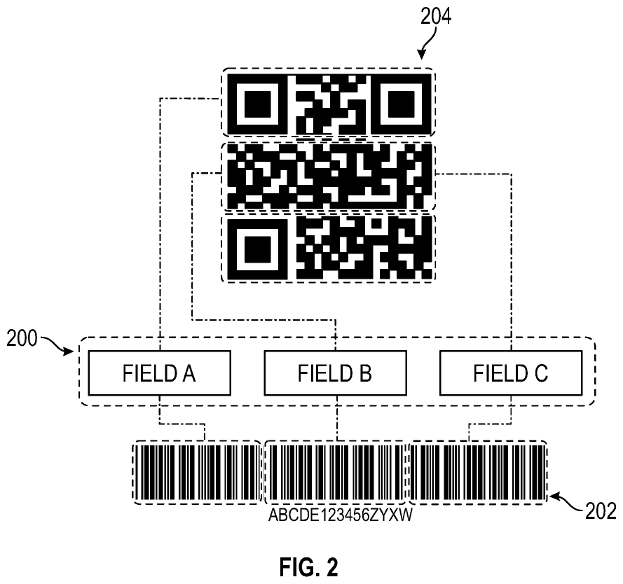

FIG. 2 depicts an example identification template 200 for identifying a medical device 12 to multiple different systems together with corresponding example displayable representations 202, 204, according to aspects of the subject technology. For the purpose of sending, receiving, and displaying identification templates or visualizations thereof, the term “medical device” or “medical device 12”, as used herein, may refer to a stand-alone medical device such as an infusion pump that does not include modules, or may refer to a PCU 14 with attached modules, or may refer to an individual module 18, 20.

The depicted template 200 is configured with multiple identifiers, each for a different system and mapped to a single medical device. According to various implementations, the template 200 may include a primary device identifier (e.g., provided by the manufacturer) and other identification information associated with a particular external (e.g., third party) system. Moreover, each template 200 may include an identifier for a care area or other location within a particular medical organization. In some implementations, each template may be assigned to the care area or location (e.g., by way of including the location identifier), and provided to the medical device and/or updated on the medical device 12 depending on the current location of the medical device.

In the depicted example, an example identification template 200 is shown with three fields (e.g., A, B, C); however, two fields or more than three fields may be provided. A field may include a first set of digits for a serial number of the device and a second set of digits for an interop (or asset) identifier. A third field may include a set of digits for a location identifier (e.g., a zip code, care area identifier, or coordinate location), and a fourth may include a set of digits for an address of the pump (e.g., an APR address). In some implementations, further information may include date information pertaining to when the device came online or service date of the device. Digits may include characters and/or numerical values. While digit fields are described, each field may take on other representation, including symbols.

Each template may be specific to a facility or a care area within a facility. In this regard, each template may be stored in a database 37 and indexed by the facility, care area, or any other identifier associated with or made part of the template. In this manner, if the device is moved to a new facility or care area, the device may receive a new template based on that facility or care area. For example, a device may move from an existing care area (e.g., a general ward) into a new care area (e.g., a neonatal intensive care unit (NICU)) and receive a template specific to the new care area. The new template is provided to the device and includes identifiers required to identify the device using systems particular to the facility or care area.

According to various implementations, the template may be further configurable by or for an entity (or entities) whose software is used to identify medical devices within the facility or healthcare organization. In this regard, the disclosed system may provide a portal interface (e.g., an Internet or network-based portal website) hosted by a server associated with the hospital organization and accessible via a terminal 32. The portal interface may allow the one or more entities (or hospital administrator) to configure templates specific to entity systems and to add information to the template in various fields. For example, Company A may wish to leverage one configuration that includes X information, while Company B may wish to have a different configuration that includes Y information. When a request is made to the system that is associated with that company, the system automatically requests the company's template. The system may then identify the virtual display template that the company requires and provide it to corresponding medical devices. When a corresponding medical device is asked to display identifying information, it may use the template to generate the identifying information. In some implementations, the template may be used to generate identifiers for provisioning to the devices, that is, the devices receive a generated identifier rather than a template for dynamic generation. In some implementations, multiple entities may associate their identifiers with a single template for a given location/care area within a hospital organization.

In some implementations, the system may provide a library of templates. An entry in the library may be associated with an entity (e.g., configured to interoperate with a system associated with the entity). An entry in the library may specify specific information elements and encoding thereof.

The system provides the information template for display as a visualization (e.g., a graphical representation displayed on a display screen). The visualization may include, for example, a linear or two-dimensional (and in some implementations three-dimensional) pattern that may be scanned and recognized and/or converted to digital information. Different devices may receive different visualizations of an identification template. For example, a first device may receive a traditional barcode visualization 202 (which can, e.g., include multiple codes displayed linearly), while a second device may receive a QR code visualization 204 having a 2D digital pattern(s) of information along an x and y axis. Both visualizations may include the particular arrangement of information of the respective template generated for a particular entity associated with the template.

When a medical device comes online or is powered on for the first time, the device may send a request to a server 30, such as a provisioning server, for an information template during a self check. The requested information may include, for example, an information template used within the device's current care area. The device may forward its serial number and current location, which the server 30 may use to index a database 37 for the template 200 corresponding to the location or an entity that services the location. The device may then make an API (application programming interface) call to the provisioning server, indicating that the device requires a template for its current location, filled in with the information specific to the area and/or the scanning software used to scan the device within the area (e.g., for asset tracking purposes). The identification server fills in the template, and a visualization of the template is provided back to the device for display on a display screen 6a or 5c of the device. A user may then scan the visualization of the template directly from the display.

In some implementations, the medical device may directly interact with the provisioning server 30 to request and receive a custom template. In some implementations, the medical device may provide its information to the provisioning server and the provisioning server may communicate with a template server (or identification server) to obtain template information, create the template, and relay a visualization of the template back to the medical device. In some implementations, the medical device may inform the template server, and the template server may provide the template back to the provisioning server, which may then distribute the template for the corresponding location to the medical device or to multiple medical devices in the corresponding location.

While the description discusses visual presentation of the templated information, in some implementations, the medical device may include a programmable and scannable wireless identifier such as a near field communication or RFID tag. In such instances, the template may identify an encoding format for a message transmitted by the scannable identifier upon activation. For example, template may define a fixed length field format whereby each field is associated with a specific data such as shown by element 200 in FIG. 2. In other implementations, the template may define a machine readable format such as XML or value separated fields or the like for presenting device identification information.

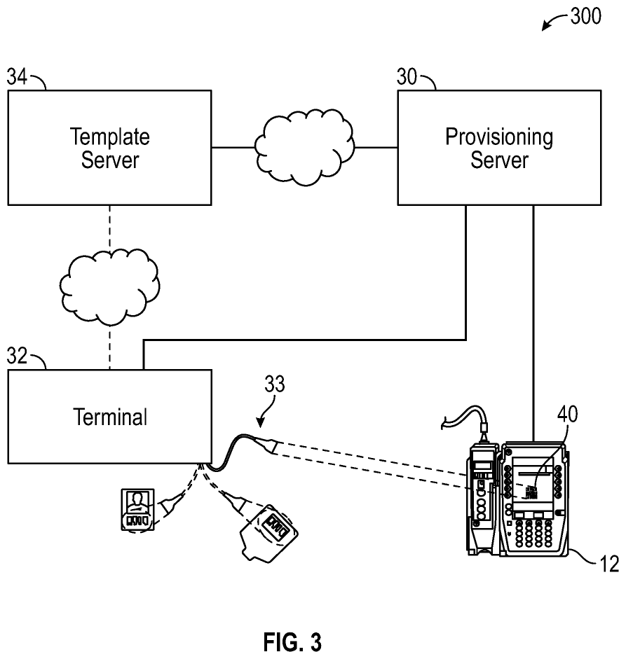

FIG. 3 depicts an example system 300 for generating and displaying an identification template for a medical device, according to aspects of the subject technology. In the depicted example, an infusion device 12 is connected to a providing server 30 and the provisioning server 30 is communicatively connected to a template server 34. The template server 34 may associate template information such as secondary identifiers with primary identifiers, and may provide a web portal interface to entities for modifying templates and associating entity created identifies with existing medical device identifiers. Terminal 32 may include a system located within a hospital organization that executes software provided by an entity to maintain, identity, and/or service medical devices within the hospital organization or a specific location or care area within the organization. In some implementations, terminal 32 is communicatively connected to the provisioning server 30 within the hospital organization. In some implementations, terminal 32 is communicatively connected to template server 34, for example, for the purpose of updating templates and/or assigning entity created/managed identifiers with existing identifiers of medical devices within the hospital organization.

In the clinical field, a user of scanning software may use a scanner 33 to scan an identifier of a medical device 12. For example, the scanner 33 may be a bar code reader (or other data input device) used to scan the coded label on the back of the device 12. In the depicted example, the identifier is displayed as a graphical visualization 40 on a display screen 6a of the device 12. According to various implementations, the visualization may be a graphical depiction of one or more identifiers, such as the displayable template 202, 204 describe with respect to FIG. 2. The reader/scanner 33 is not required to be integrated with the medical device 12. The scanner 33 may be part of a separate device such as an interoperability terminal 32 (e.g., part of one or more computing devices), which may be connected to the same network 40 as the infusion device 12 and configured with software to function in an overall workflow involving the infusion device 12.

The scanning may be related to an inventory process for maintaining and/or tracking assets of a hospital organization. In this regard, the scanned information may be used to store and/or associate a scanned identifier with asset tracking information, or to look up asset information based on the scanned identifier. The scanning may be for associating an infusion device with other devices such as a medication to be administered by the device or with a patient designated to receive it. The scanning may be related to programming the device or for identifying the device to an EMR (electronic medical records) system to send an automated programming request (APR) to the medical device 12, for example, to load parameters pertaining to an order.

While the process may be described herein with respect to a provisioning server 30 and a template server 34, the provisioning server and template server may exist as programming modules on a single server or computing system. The messages transmitted between the terminal 32, the medical device 12, and the servers 30, 34 may be routed differently and/or the process may be performed with multiple or only one server system.

According to various implementations, a user initiates a display of a scannable identifier on the medical device 12 to initiate a scanning activity. The identifier may be activated for scanning by activating a control 6b or 6c on the medical device. While the subject technology is described herein with reference to a medical device 12, for example with a patient control unit 14, the medical device may include other devices such as dispensing cabinets and devices, or may be modules connected to a PCU 14. For example, the user may activate a control 5e-h on a medical device module 18, 20 and interact with and/or scan an identifier displayed on display 5c.

The scanning may initiate a process by which information pertaining to the device (e.g., a code affixed to the device) is scanned by the scanner 33 and is automatically sent to a centralized server via a network 40. As will be described further, when the disclosed identifier is scanned by a scanner 33 the terminal 32 may determine whether the scanned information contains an identifier that is compatible with an external system. In some implementations, the external system may include software running on the terminal or the server 34. In some implementations, the terminal determines whether the identifier is in a format compatible with the external system or a format that corresponds to the current area of the device 12. For example, a first format may be required for a general ward and a second format may be required for a NICU. If the scanning takes place in the NICU and the scanning system (e.g., terminal 32) determines that the scanned identifier is in the first format then the system may request that the identifier be updated to the second format. When the scanning system determines that an update is required, the terminal 32 may obtain a serial number of the device via the scan or via manual input and push up the serial number to server 30, 34 with a request to update the device with a new identifier for the current location.

Provisioning server 30 receives a message indicating that the medical device is ready to receive an identification template. In some implementations, the message may be sent by the device 12 on activation of the device (e.g., when powered on and/or activated for the first time). The message may include, for example, an internal identifier for the device 12 (e.g., a serial number). In some implementations, input at the terminal 32 may indicate the internal identifier for the device and that the device is in need of an identification template corresponding to the location of the terminal and/or device, or corresponding to the software system running on the terminal (e.g., an asset management software for a particular asset management vendor). In some implementations, the message may include a request for a template that includes information for all entities known to the location (e.g., all companies associated with a service area).

The provisioning server 30 may determine whether a template is available for the medical device based on a current location of the medical device. The location may be determined by the server based on location data received with the message, or the server 30 may know the location of the medical device based on the current wireless node the medical device is connected to, a care area entered into the medical device, a care area associated with a clinician that logged into the device (e.g., by scanning an RFID badge), or by querying an EMR server which receives location data with other data as the medical device is used within the hospital organization. If a template is not available (e.g., for the location) then the server 30 may generate the identification template based on provisioning information received from the template server 34. In some implementations, server 34 may generate the template and provide the template to server 30.

According to various implementations, provisioning server 30 receives multiple identification identifiers (or tags) for a medical device and generates the template described with regard to FIG. 2. The template may include an identifier that corresponds to a single entity or may include multiple identifiers for entities known to service a given location. The server then generates a visualization of the template for display on a display screen of the medical device and forwards the visualization to the medical device for display, as needed. When the medical device receives the visualization it may be immediately displayed as part of a single workflow (e.g., including the request) or may be activated by the user selecting the appropriate control 6b, 6c. Once displayed, the scanning device 33 may be used to scan the visualization to selectively read the identifier that corresponds to the software executing in conjunction with the scanning device.

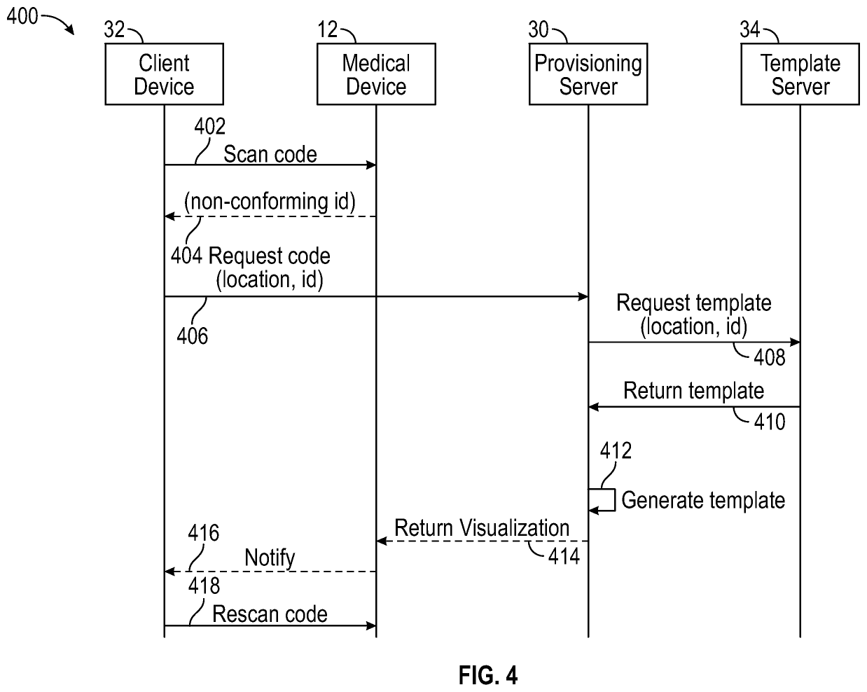

FIG. 4 depicts an example process flow diagram 400 for an automated asset identification system. For explanatory purposes, the various blocks of example process flow 400 are described herein with reference to FIGS. 1A, 1B, 2, and 3, and the associated components and/or processes described herein. The depicted process is directed to a terminal for an asset scanning system initiating a request to receive a visualization for an identification template. However, as described previously and further below, the request may be sent by other systems such as from the medical device or an associated terminal of the medical device (e.g., a mobile device connected to the medical device). The lines represent one or more messages that may be generated, communicated or both between the illustrative entities in FIG. 4.

In the depicted example, a client device 32 scans a visualization 40 of an identification template 200 which is displayed on a display 6a, 5c of a medical device 12 (402). Under normal operating circumstances, a field of the template 200 includes an identifier expected by an external system associated with a client device 32 (or software thereon). When a non-confirming identifier is received/scanned (404), the software may request an updated template for the medical device (406). In this regard, the client device 32 sends a request (406) to the provisioning server 30, for the server to update the medical device with a new template for the client device. The request includes identifying information for the infusion device such as a location of the device and/or the non-conforming identifier (if received) from the previously scanned template visualization. Additionally or in the alternative, as described previously, the request may be sent from the medical device 12 by the user activating a control on the medical device to update the template.

The provisioning server 30 requests a template for the current location of the medical device 12 (408). In this regard, the server 30 may query a template server for provisioning information based on the location of the medical device. The template server 34 then returns provisioning information corresponding to the location of the medical device (410). The provisioning information may include, for example, a default template corresponding to the location of the medical device. In some implementations, the default template may include a second identifier (e.g., an asset identifier) corresponding for the device. In some implementations, the provisioning server 30 may generate the second identifier.

The provisioning server 30 generates the final template for the medical device (412) based on the first identifier and the provisioning information. In some implementations, the server 30 appends a default template with the first identifier of the medical device and/or the second identifier. The server then generates a visualization 40 of the template and returns the template visualization to the medical device (414). The medical device may display an alert or present the visualization when it receives the updated template, thereby notifying the clinician (416). In some implementations, the provisioning server 30 may notify the client device 32 directly when the visualization is made available to the medical device. The user may then activate or access the medical device to display the visualization 40 and rescan the updated visualization (418).

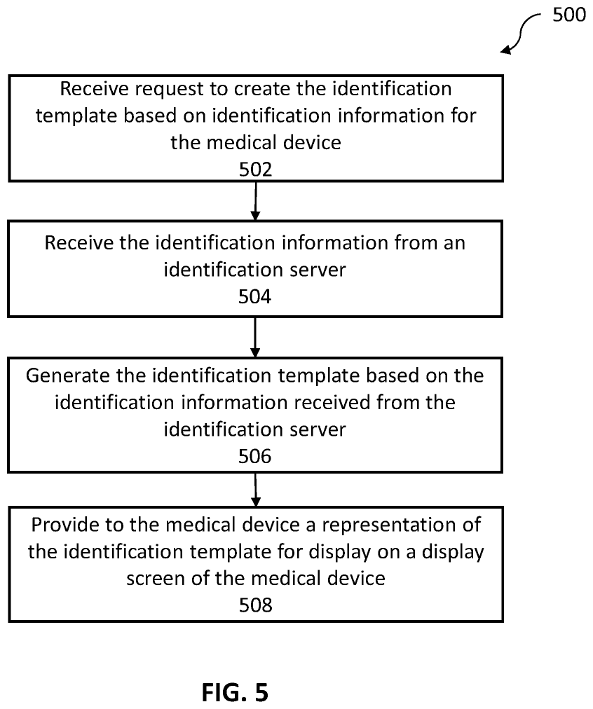

FIG. 5 depicts a first example process 500 for automatically programming a medical device, according to aspects of the subject technology. For explanatory purposes, the various blocks of example process 500 are described herein with reference to FIGS. 1A, 1B, 2, 3 and 4, and the associated components and/or processes described herein. The one or more of the blocks of process 500 may be implemented, for example, by one or more computing devices including, for example, provisioning server 30, template server 34, or a client computing device 32. In some implementations, one or more of the blocks may be implemented based on one or more machine learning algorithms. In some implementations, one or more of the blocks may be implemented apart from other blocks, and by one or more different processors or devices. Further, for explanatory purposes, to the extent that the blocks of example process 500 are described as occurring in serial, or linearly, in some implementations, multiple blocks of example process 500 may occur in parallel (e.g., blocks 502 and 504 may occur in parallel). In addition, the blocks of example process 500 need not be performed in the order shown and/or one or more of the blocks of example process 500 need not be performed.

According to various implementations, a provisioning server receives a request to create an identification template for a medical device based on a first identifier currently assigned to the medical device and provisioning information obtained from an identification server (502). In some implementations, the request originates from the medical device 12.

A user may activate the device to initiate a scanning of a virtual identifier. For example, the user may be undertaking asset tracking or maintenance of the device and require scanning a third party identifier associated with the device for that purpose. If no virtual identifier is currently stored on the device, the device may send a message to the provisioning server 30 indicating that the device is ready to receive an identification template 200. If a template 200 is available, the server sends a visualization 40 of the template to the device and the device displays visualization to be scanned by a scanning device 33. If the template is not available, the server provides an indication that the template is currently not available for the device. The device may then send the request to create the template 200.

In some implementations, the medical device 12 may display a default visualization of an identifier, which is then scanned by the user using a scanner 33. The scanning software may be part of an external system, and may determine, on scanning the visualization 40, that the template does not include an identifier expected by the external system (or software thereof) and/or that does not correspond to the current location of the medical device. Accordingly, the scanning software may present an alert regarding the missing identifier at a display of the scanning terminal 32. In some implementations, the scanning software may notify the provisioning server 30 that the medical device is in need of a new template. The scanning software may identify the medical device to the provisioning server 30 using an identifier read from the current visualization, or may provide the visualization to the provisioning server, and the server may determine the medical device based on identification information within the template corresponding to the visualization.

On receiving the request to create the identification template, the server 30 obtains provisioning information from a template server 34 (504). According to various implementations, the provisioning information includes identification information that pertains to a particular location of the medical device and/or to the entity for which a secondary identification (e.g., other than the device's primary identification) is needed. The entity may be a company, for example, a vendor account associated with asset management. The provisioning information may be stored in a database 37 and obtained by querying/indexing the database based on a location of the medical device and/or an identifier associated with the entity.

On obtaining the provisioning information, the provisioning server 30 generates the requested identification template based on the first identifier and the provisioning information (506). As described previously, the identification template may include an arrangement of multiple codes (e.g., two, three or more). One code may include the primary identifier for the device, such as a manufacturer serial number, while a second code may include an identifier specific to the location and/or entity. Generating the template may include the server determining an arrangement and values of the codes based on predetermined settings associated with an account associated with a given entity (e.g., a vendor associated with asset management).

After the requested identification template has been generated, the provisioning server 30 sends a visualization of the template to the medical device 12. According to various implementations, the visualization may be a graphic image displayed on a display screen. In some implementations, the visualization is a barcode 202 or QR code 204, with multiple fields embedded therein. In this regard, when scanned, the scanning device 33 may parse the visualization and determine the code it needs. For example, a first scanning software package for a first entity may scan the visualization and utilize the code/identifier in a first field of the visualization, while a second scanning software package associated with a second entity may scan the same visualization and utilize the code/identifier in a second filed of the visualization.

As described previously, the disclosed identification template 200 may be specific to a location such as a specific facility or a care area within a hospital organization. For example, a hospital organization may assign a specific vendor or vendor software for asset management to a given care area or facility location, and the identification templates may be used by the vendors to select their own identification schemes. In this regard, the system may select a new template for a device as it moves throughout a hospital organization, depending on which vendors service each area of the organization. The system may be configured to update a device's template when the device moves from one location to another (e.g., from a first care unit to a second care unit). When a device 12 moves within the organization and changes care area or facility, the device may report the new care area to the server 30. The server 30 receives an indication that the device moved from a first location to a second location, and generates, responsive to receiving the indication, an updated identification template based on the second location. The server 30 may then push the visualization to the medical device or otherwise cause the medical device to update the visualization with the updated template.

In some implementations, an entity may request to have multiple medical devices updated with an identification template that includes an identity for the entity. In this regard, a user for an entity may log on to the provisioning server 30 (or template server 34) via a portal website and request a list of medical devices associated with a care area or location. The list may then be presented to the user who may select one or multiple medical devices from the list to receive the new template. Identifiers for the selected medical devices are provided to the template server 34, which assigns information to the identifiers on behalf of the entity. For example, the template server may assign provisioning information, including secondary (or asset) identifiers associated with the entity to each of the medical devices. The provisioning server then receives, from the template server, the provisioning information for each of the medical devices, and then provides each respective medical device a respective visualization the generated identification template. The provisioning server may push out the visualizations over the hospital's device network 40, or the visualizations may be provided when requested by the devices as described previously herein.

Many of the above-described example process 500 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

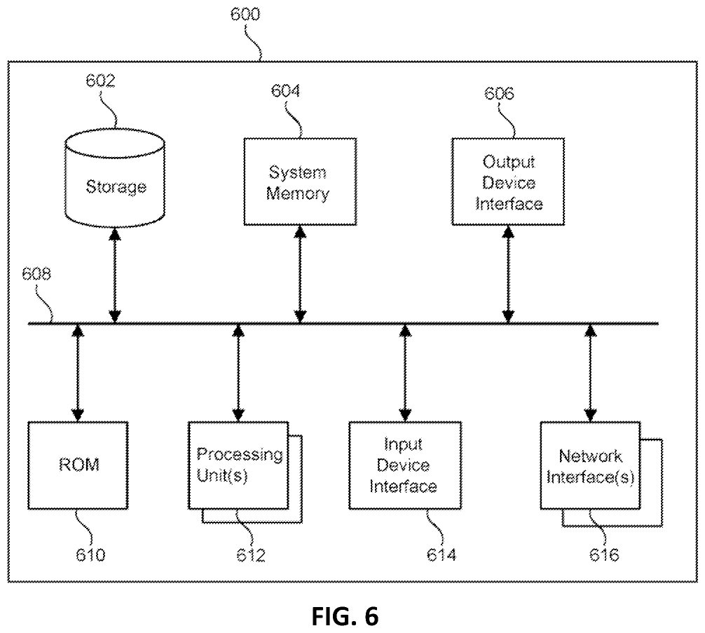

FIG. 6 is a conceptual diagram illustrating an example electronic system 600 for automatically programming a medical device, according to aspects of the subject technology. Electronic system 600 may be a computing device for execution of software associated with one or more portions or steps of processes 300, 350, 400, and 500 or components and methods provided by FIGS. 1-5, including but not limited to computing hardware within servers 30, 34, terminal 32, 206, medical device 12, and/or any computing devices or associated modules or terminals disclosed herein. Electronic system 600 may be a specifically configured personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or similar computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry specifically configured for operation of the various components and methods previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as, random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 408 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware or network interface module) for connecting the computer to a part of a network of computers such as a local area network (“LAN”), a wide area network (“WAN”), wireless LAN, or an Intranet, or a network of networks, such as the Internet. Any or all components of electronic system 600 can be specifically configured to be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification

Clause 1. A medical information system, comprising: one or more computing devices configured to: receive a request to create an identification template for a medical device based on a first identifier currently assigned to the medical device and provisioning information obtained from an identification server; receive the provisioning information based on the request; generate the identification template based on the first identifier and the provisioning information; and provide to the medical device, responsive to generating the identification template, a visualization of the identification template for display on a display screen of the medical device.

Clause 2. The medical information system of Clause 1, wherein the one or more computing devices are configured to: receive, before receiving the request to create the identification template, a message indicating that the medical device is ready to receive the identification template; provide, to the medical device, responsive to the message, an indication that the identification template is not available for the medical device; and receive the request to create the identification template from the medical device after providing the indication that the identification template is not available.

Clause 3. The medical information system of Clause 1 or Clause 2, wherein the one or more computing devices are configured to: receive an indication that a default visualization of an identifier was scanned by a client device and that the default visualization does not include a second identifier required by the client device; generate the identification template responsive to the indication, wherein the provisioning information includes the second identifier and the identification template is generated based on a location of the medical device and the second identifier; and receive an indication that the visualization was scanned by the client device.

Clause 4. The medical information system of Clause 1 or Clause 2, further comprising: non-transitory computer readable storage medium having identification scanning software stored thereon that, when executed by the identification server, cause the identification server to: receive, from the medical device, the request to create the identification template for the medical device, the request including the first identifier of the medical device; and associating a second identifier with the medical device and scanning software associated with the identification server, the second identifier being different than the first identifier, wherein the provisioning information includes the second identifier and the identification template is generated based on the second identifier, and wherein generating the identification template further comprises generating the identification template based on the second identifier.

Clause 5. The medical information system of any one of Clauses 1 through 4, wherein the one or more computing devices are further configured to: receive, from the medical device, a first location of the medical device, wherein the provisioning information from the identification server comprises a second identifier associated with the medical device; and generate the identification template based on the first identifier, the second identifier, and the received first location of the medical device.

Clause 6. The medical information system of Clause 5, wherein the one or more computing devices are further configured to: receive an indication that the medical device moved to a second location; generate, responsive to receiving the indication that the medical device moved to the second location, an updated identification template based on the second location of the medical device; and cause the medical device to update the visualization with the updated identification template.

Clause 7. The medical information system of any one of Clauses 1 through 6, wherein the one or more computing devices are further configured to: receive a location of the medical device and an indication that the medical device has been powered on for the first time; and receive the provisioning information for the medical device based on querying a data system based on the received location of the medical device.

Clause 8. The medical information system of any one of Clauses 1 through 7, wherein the one or more computing devices are further configured to: receive, from an external system, a request for a list of medical devices associated with a care area; provide the list of medical devices to the external system in response to the request; receive, from the external system, provisioning information for each of the medical devices, the provisioning information including asset identifiers for each medical device in the list of medical devices;

and provide to each respective medical device of the medical devices a respective visualization for an identification template that includes an asset identifier for the respective medical device.

Clause 9. The medical information system of any one of Clauses 1 though 8, wherein the visualization is a graphic image displayed on the display screen.

Clause 10. The medical information system of any one of Clauses 1 though 9, wherein the identification template comprises an arrangement of a three or more codes, and wherein generating the identification template comprises: identifying a vendor account designated to scan the visualization; determining the arrangement and values of the three or more codes based on predetermined settings associated with the vendor account.

Clause 11. A machine-implemented method for an automated asset identification system, comprising: receiving a request to create an identification template for a medical device based on a first identifier currently assigned to the medical device and provisioning information obtained from an identification server; receiving the provisioning information based on the request; generating the identification template based on the first identifier and the provisioning information; and providing to the medical device, responsive to generating the identification template, a visualization of the identification template for display on a display screen of the medical device.

Clause 12. The machine-implemented method of Clause 11, further comprising: receiving, before receiving the request to create the identification template, a message indicating that the medical device is ready to receive the identification template; providing, to the medical device, responsive to the message, an indication that the identification template is not available for the medical device; and receiving the request to create the identification template from the medical device after providing the indication that the identification template is not available.

Clause 13. The machine-implemented method of Clause 11 or Clause 12, further comprising: receiving an indication that a default visualization of an identifier was scanned by a client device and that the default visualization does not include a second identifier required by the client device; generating the identification template responsive to the indication, wherein the provisioning information includes the second identifier and the identification template is generated based on a location of the medical device and the second identifier; and receiving an indication that the visualization was scanned by the client device.

Clause 14. The machine-implemented method of Clause 11 or Clause 12, further comprising: receiving, from the medical device, the request to create the identification template for the medical device, the request including the first identifier of the medical device; and associating a second identifier with the medical device and scanning software associated with an external system, the second identifier being different than the first identifier, wherein the provisioning information includes the second identifier and the identification template is generated based on the second identifier, and wherein generating the identification template further comprises generating the identification template based on the second identifier.

Clause 15. The machine-implemented method of Clause 11, further comprising: receiving, from the medical device, a first location of the medical device, wherein the provisioning information from the identification server comprises a second identifier associated with the medical device; and generating the identification template based on the first identifier, the second identifier, and the received first location of the medical device.

Clause 16. The machine-implemented method of Clause 15, further comprising: receiving an indication that the medical device moved to a second location; generating, responsive to receiving the indication that the medical device moved to the second location, an updated identification template based on the second location of the medical device; and causing the medical device to update the visualization with the updated identification template.

Clause 17. The machine-implemented method of any one of Clauses 11 through 17, further comprising: receiving a location of the medical device and an indication that the medical device has been powered on for the first time; and receiving the provisioning information for the medical device based on querying a data system based on the received location of the medical device.

Clause 18. The machine-implemented method of any one of Clauses 11 through 17, further comprising: receiving, from an external system, a request for a list of medical devices associated with a care area; providing the list of medical devices to the external system in response to the request; receiving, from the external system, provisioning information for each of the medical devices, the provisioning information including asset identifiers for each medical device in the list of medical devices; and provide to each respective medical device of the medical devices a respective visualization for an identification template that includes an asset identifier for the respective medical device.

Clause 19. The medical information system of any one of Clauses 11 through 18, wherein the identification template comprises an arrangement of a three or more codes, and wherein generating the identification template comprises: identifying a vendor account designated to scan the visualization; determining the arrangement and values of the three or more codes based on predetermined settings associated with the vendor account.

Clause 20. A non-transitory computer readable medium comprising instructions that, when executed by a computing system, cause the computing system to perform a method according to any one of Clauses 11 through 19.

### Further Considerations

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” when used to describe a relationship between two or more elements encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A medical information system, comprising:
one or more computing devices configured to:
receive a request to create an identification template for a medical device based on a first identifier currently assigned to the medical device and provisioning information obtained from an identification server;
receive the provisioning information based on the request;
generate the identification template based on the first identifier and the provisioning information; and
provide to the medical device, responsive to generating the identification template, a visualization of the identification template for display on a display screen of the medical device.

2. The medical information system of claim 1, wherein the one or more computing devices are configured to:
receive, before receiving the request to create the identification template, a message indicating that the medical device is ready to receive the identification template;
provide, to the medical device, responsive to the message, an indication that the identification template is not available for the medical device; and
receive the request to create the identification template from the medical device after providing the indication that the identification template is not available.

3. The medical information system of claim 1, wherein the one or more computing devices are configured to:
receive an indication that a default visualization of an identifier was scanned by a client device and that the default visualization does not include a second identifier required by the client device;
generate the identification template responsive to the indication, wherein the provisioning information includes the second identifier and the identification template is generated based on a location of the medical device and the second identifier; and
receive an indication that the visualization was scanned by the client device.

4. The medical information system of claim 1, further comprising:
non-transitory computer readable storage medium having identification scanning software stored thereon that, when executed by the identification server, cause the identification server to:
receive, from the medical device, the request to create the identification template for the medical device, the request including the first identifier of the medical device; and
associating a second identifier with the medical device and scanning software associated with the identification server, the second identifier being different than the first identifier,
wherein the provisioning information includes the second identifier and the identification template is generated based on the second identifier, and
wherein generating the identification template further comprises generating the identification template based on the second identifier.

5. The medical information system of claim 1, wherein the one or more computing devices are further configured to:
receive, from the medical device, a first location of the medical device, wherein the provisioning information from the identification server comprises a second identifier associated with the medical device; and
generate the identification template based on the first identifier, the second identifier, and the received first location of the medical device.

6. The medical information system of claim 5, wherein the one or more computing devices are further configured to:
receive an indication that the medical device moved to a second location;
generate, responsive to receiving the indication that the medical device moved to the second location, an updated identification template based on the second location of the medical device; and
cause the medical device to update the visualization with the updated identification template.

7. The medical information system of claim 1, wherein the one or more computing devices are further configured to:
receive a location of the medical device and an indication that the medical device has been powered on for the first time; and
receive the provisioning information for the medical device based on querying a data system based on the received location of the medical device.

8. The medical information system of claim 1, wherein the one or more computing devices are further configured to:
receive, from an external system, a request for a list of medical devices associated with a care area;
provide the list of medical devices to the external system in response to the request;
receive, from the external system, provisioning information for each of the medical devices, the provisioning information including asset identifiers for each medical device in the list of medical devices; and
provide to each respective medical device of the medical devices a respective visualization for an identification template that includes an asset identifier for the respective medical device.

9. The medical information system of claim 1, wherein the visualization is a graphic image displayed on the display screen.

10. The medical information system of claim 1, wherein the identification template comprises an arrangement of a three or more codes, and wherein generating the identification template comprises:
identifying a vendor account designated to scan the visualization;
determining the arrangement and values of the three or more codes based on predetermined settings associated with the vendor account.

11. A machine-implemented method for an automated asset identification system, comprising:
receiving a request to create an identification template for a medical device based on a first identifier currently assigned to the medical device and provisioning information obtained from an identification server;
receiving the provisioning information based on the request;
generating the identification template based on the first identifier and the provisioning information; and
providing to the medical device, responsive to generating the identification template, a visualization of the identification template for display on a display screen of the medical device.

12. The machine-implemented method of claim 11, further comprising:
receiving, before receiving the request to create the identification template, a message indicating that the medical device is ready to receive the identification template;
providing, to the medical device, responsive to the message, an indication that the identification template is not available for the medical device; and
receiving the request to create the identification template from the medical device after providing the indication that the identification template is not available.

13. The machine-implemented method of claim 11, further comprising:
receiving an indication that a default visualization of an identifier was scanned by a client device and that the default visualization does not include a second identifier required by the client device;
generating the identification template responsive to the indication, wherein the provisioning information includes the second identifier and the identification template is generated based on a location of the medical device and the second identifier; and
receiving an indication that the visualization was scanned by the client device.

14. The machine-implemented method of claim 11, further comprising:
receiving, from the medical device, the request to create the identification template for the medical device, the request including the first identifier of the medical device; and
associating a second identifier with the medical device and scanning software associated with an external system, the second identifier being different than the first identifier,
wherein the provisioning information includes the second identifier and the identification template is generated based on the second identifier, and
wherein generating the identification template further comprises generating the identification template based on the second identifier.

15. The machine-implemented method of claim 11, further comprising:
receiving, from the medical device, a first location of the medical device, wherein the provisioning information from the identification server comprises a second identifier associated with the medical device; and
generating the identification template based on the first identifier, the second identifier, and the received first location of the medical device.

16. The machine-implemented method of claim 15, further comprising:
receiving an indication that the medical device moved to a second location;
generating, responsive to receiving the indication that the medical device moved to the second location, an updated identification template based on the second location of the medical device; and
causing the medical device to update the visualization with the updated identification template.

17. The machine-implemented method of claim 11, further comprising:
receiving a location of the medical device and an indication that the medical device has been powered on for the first time; and
receiving the provisioning information for the medical device based on querying a data system based on the received location of the medical device.

18. The machine-implemented method of claim 11, further comprising:
receiving, from an external system, a request for a list of medical devices associated with a care area;
providing the list of medical devices to the external system in response to the request;
receiving, from the external system, provisioning information for each of the medical devices, the provisioning information including asset identifiers for each medical device in the list of medical devices; and
provide to each respective medical device of the medical devices a respective visualization for an identification template that includes an asset identifier for the respective medical device.

19. The medical information system of claim 11, wherein the identification template comprises an arrangement of a three or more codes, and wherein generating the identification template comprises:
identifying a vendor account designated to scan the visualization;
determining the arrangement and values of the three or more codes based on predetermined settings associated with the vendor account.

20. A non-transitory computer readable medium comprising instructions that, when executed by a computing system, cause the computing system to perform a method according to claim 11.
